(19) United States
(12) Patent Application Publication
(10) Pub. No.: US 2022/0288019 A1
(43) Pub. Date: Sep. 15, 2022

(54) Methods of treating cancer using a combination of SERD Dosing Regimens

(71) Applicant: Eli Lilly and Company, Indianapolis, IN (US)

(72) Inventors: Shripad Venkatraman BHAGWAT, Carmel, IN (US); Lillian Mary SMYTH, Dublin (IE); Suzanne Rebecca Lou YOUNG, Greenfield, IN (US); Natalie Starr HOLMAN, Indianapolis, IN (US); Daniel Jon SALL, Greenwood, IN (US); Safi SHAHDA, Indianapolis, IN (US); Eunice Soek Mun YUEN, Ashford Middlesex (GB)

(21) Appl. No.: 17/689,286

(22) Filed: Mar. 8, 2022

Related U.S. Application Data
(60) Provisional application No. 63/281,143, filed on Nov. 19, 2021.
(60) Provisional application No. 63/158,688, filed on Mar. 9, 2021.

(51) Int. Cl. A61K 31/397; A61P 35/04; A61K 39/395; A61K 45/06; A61K 31/4196; A61K 31/566

(52) U.S. Cl. CPC A61K 31/397; A61P 35/04; A61K 39/39558; A61K 45/06; A61K 31/4196; A61K 31/566

(57) ABSTRACT

Disclosed herein are dosing regimens for the administration of a compound of Formula I:

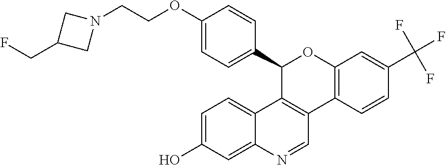 [SMILES: Oc1ccc2c3c(cnc2c1)-c1ccc(C(F)(F)F)cc1O[C@@H]3c1ccc(OCCN2CC(CF)C2)cc1]

or pharmaceutically acceptable salt thereof to patients in need of such treatment. The dosing regimens include adjuvant therapy.

### CROSS REFERENCE

This application incorporates by reference U.S. Provisional Application Ser. No. 63/158,688, filed Mar. 9, 2021, in its entirety, for all purposes.

### FIELD

This disclosure is directed to the fields of cancer treatment.

### BACKGROUND

Selective estrogen receptor degraders (SERDs) bind to the estrogen receptor (ER) and downregulate ER-mediated transcriptional activity. This degradation and downregulation caused by SERDs can be useful in the treatment of cell proliferation disorders, such as cancer.

Drug development is unpredictable. It is common for new molecules to fail in preclinical and/or clinical stages, often for little understood reasons. Dosing is an added complexity that drives further unpredictability. Not all drug dosing regimens are equally active in all patient populations due to various factors, including but not limited to, body weight, performance status, number of prior systemic therapies, genetics, and histological tumor type. Toxicity issues introduce additional complexities. Efficacy and toxicity must be balanced. Additionally, it is not always possible to predict which patients will achieve therapeutic serum levels fast enough to receive benefit before disease progression. Administering a loading dose seeks to provide possible remediation of early progression in the previously non-responding subpopulation without resulting in toxicological barriers for that population or the previously responding population.

There remains a need to provide alternative treatment therapies for patients suffering from cancer. In addition, there remains a need to provide alternative treatment therapies that possess better tolerability profiles, or to provide alternative therapies that allow for maximum activity with limited adverse events and fewer dose interruptions or discontinuations. There remains a need for potent anti-estrogen treatments that antagonize and degrade ER with clinically relevant activity and bioavailability. (Shagufta, et al., Recent progress in selective estrogen receptor down regulators (SERDs) for the treatment of breast cancer, RSC Med. Chem., 2020, 11, 438-454.)

### SUMMARY

The present disclosure relates to new dosing protocols that use (5R)-5-[4-[2-[3-(fluoromethyl)azetidin-1-yl]ethoxy]phenyl]-8-(trifluoromethyl)-5H-chromeno[4,3-c]quinolin-2-ol, hereafter known as Formula I, or a pharmaceutically acceptable salt thereof, as part of an adjuvant therapy, to treat cancer.

The compound of Formula I has the following structure:

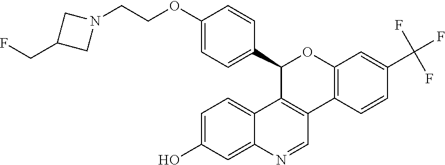 [SMILES: Oc1ccc2c3c(cnc2c1)-c1ccc(C(F)(F)F)cc1O[C@@H]3c1ccc(OCCN2CC(CF)C2)cc1]

or pharmaceutically acceptable salt thereof.
This compound may be prepared as a free base or a pharmaceutically acceptable salt thereof, using the synthetic steps described in WO20/014435 or U.S. Pat. No. 10,654,866. This compound is known by the tradename “imlunestrant.”

The compound of Formula I is an orally bioavailable, selective SERD. It is a potent degrader and selective antagonist of wild-type and mutant estrogen receptor α (ERα or ESR1). It would be useful to develop new treatment regimens and dosing protocols that use the compound of Formula I, in combination with one or more other therapeutic agents, in conjunction with surgery, or in combination with one or more other therapeutic agents and in conjunction with surgery, as part of an adjuvant therapy, to treat cancer.

Disclosed herein are dosing protocols that use the compound of Formula Ito treat cancer. The protocols may be monotherapy or adjuvant therapy.

In an aspect, the dosing protocols comprise methods of treating a cancer comprising: administering a dose between about 200 mg and about 800 mg of a compound of Formula I:

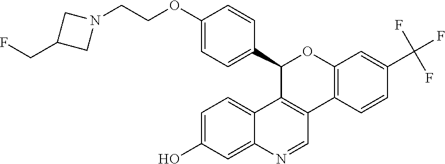 [SMILES: Oc1ccc2c3c(cnc2c1)-c1ccc(C(F)(F)F)cc1O[C@@H]3c1ccc(OCCN2CC(CF)C2)cc1]

or pharmaceutically acceptable salt thereof, to a patient in need of such treatment, at least once a day.

In another aspect, the dosing protocols comprise methods of treating a cancer comprising:

administering a dose between about 200 mg and about 400 mg of a compound of Formula I: or pharmaceutically acceptable salt thereof, to a patient in need of such treatment, at least once a day for at least one week.

In an aspect, the dosing protocols comprise methods of treating a cancer comprising: administering a dose of about 200 mg of the compound of Formula I or pharmaceutically acceptable salt thereof, at least once a day, to a patient in need of such treatment, for at least about one week and then increasing the dose to about 300 mg or about 400 mg at least once a day.

In an aspect, the dosing protocols comprise methods of treating a cancer comprising: administering a dose of about 200 mg of the compound of Formula I or pharmaceutically acceptable salt thereof,

at least once a day, to a patient in need of such treatment, for about two weeks to about six months, and then increasing the dose to about 300 mg at least once a day, for at least 21 days.

In an aspect, the dosing protocols comprise methods of treating a cancer comprising: administering a dose of about 200 mg of the compound of Formula I or pharmaceutically acceptable salt thereof, at least once a day, to a patient in need of such treatment, for about two weeks to about six months, and then increasing the dose to about 400 mg at least once a day, for at least 21 days.

In another aspect, the dosing protocols comprise methods of treating a cancer comprising administering a dose of about 400 mg of the compound of Formula I: or pharmaceutically acceptable salt thereof, at least once a day, to a patient in need of such treatment.

In an aspect, the dosing protocols comprise methods of treating breast cancer, the methods comprising administering a dose of about 400 mg of the compound of Formula I or pharmaceutically acceptable salt thereof, at least once a day, to a patient in need of such treatment.

In an aspect, the dosing protocols comprise methods of treating breast cancer, the methods comprising administering a dose of about 400 mg of the compound of Formula I or pharmaceutically acceptable salt thereof,

at least once a day, to a patient in need of such treatment, for at least about one week to about six months, and then decreasing the dose to about 200 mg at least once a day.

In an aspect, the dosing protocols comprise methods of treating a cancer comprising administering a dose of about 400 mg of the compound of Formula I or pharmaceutically acceptable salt thereof, at least once a day, to a patient in need of such treatment, for about two weeks to about six months, and then decreasing the dose to about 200 mg at least once a day for at least 21 days.

In an aspect, the dosing protocols comprise methods of treating a cancer comprising administering the compound of Formula I or pharmaceutically acceptable salt thereof, in combination with a second therapeutic agent for the treatment of a cancer in a patient, the cancer selected from the group consisting of: breast cancer including metastatic breast cancer (mBC), advanced breast cancer, ovarian cancer, endometrial cancer, including endometrioid endometrial cancer (EEC), prostate cancer, uterine cancer, gastric cancer, and lung cancer, wherein the compound of Formula I or a pharmaceutically acceptable salt thereof is administered at a dose between about 200 mg and about 400 mg.

In an aspect, the dosing protocols comprise methods of treating a cancer comprising administering a dose of about 400 mg of the compound of Formula I or pharmaceutically acceptable salt thereof, at least once a day, to a patient in need of such treatment.

In an aspect, disclosed herein is a method of treating a cancer comprising: administering a dose between about 200 mg and about 400 mg of a compound of Formula I or pharmaceutically acceptable salt thereof, a second therapeutic, and a third therapeutic, wherein the second therapeutic and the third therapeutic are different, to a patient in need of such treatment, at least once a day for at least one week.

In an aspect, disclosed herein is a method of treating breast cancer, the method comprising administering a dose of about 400 mg of the compound of Formula I or pharmaceutically acceptable salt thereof, at least once a day, to a patient in need of such treatment.

In an aspect, the dosing protocols comprise methods of treating ER+, HER2-negative breast cancer, the method comprising administering (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1), pertuzumab and trastuzumab.

In another aspect, the dosing protocols comprise methods of treating ER+, HER2-positive breast cancer, the method comprising administering (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1), pertuzumab and trastuzumab.

In an aspect, the dosing protocols comprise methods of treating ER+, HER2-negative breast cancer, the method comprising administering 400 mg of (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1), 6 mg/kg of pertuzumab (Q21D) and 420 mg trastuzumab (Q21D), where Q21D means every 21 days.

In another aspect, the dosing protocols comprise methods of treating ER+, HER2-positive breast cancer, the method comprising administering (5R)-5-[4-[2-[3-(fluoromethyl)azetidin-1-yl] ethoxy]phenyl]-8-(trifluoromethyl)-5H-chromeno[4,3-c]quinolin-2-ol, 4-methylbenzenesulfonic acid (1/1), 6 mg/kg of pertuzumab (Q21D) and 420 mg trastuzumab (Q21D).

In an aspect, the dosing protocols comprise methods of treating ER+, HER2-negative breast cancer, the method comprising administering about 400 mg at least once a day of (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1), and abemaciclib.

In an aspect, the dosing protocols comprise a compound of Formula I or pharmaceutically acceptable salt thereof, for use in treating cancer, wherein the compound, or pharmaceutically acceptable salt thereof, is administered to a patient at a dose between about 200 mg and about 800 mg at least once a day for at least a week.

In another aspect, the dosing protocols comprise a compound of Formula I or pharmaceutically acceptable salt thereof for use in treating cancer, wherein the compound, or pharmaceutically acceptable salt thereof, is administered to a patient at a dose of about 200 mg at least once a day for about two weeks to about six months, and then the dose is increased to about 300 mg at least once a day for at least 21 days.

In an aspect, the dosing protocols comprising a compound of Formula I or pharmaceutically acceptable salt thereof, for use in treating cancer, wherein the compound, or pharmaceutically acceptable salt thereof, is administered to a patient at a dose of about 200 mg at least once a day for about two weeks to about six months, and then the dose is increased to about 400 mg at least once a day for at least 21 days. In an aspect, the dosing protocols comprisea compound of Formula I or pharmaceutically acceptable salt thereof, for use in treating cancer, wherein the compound, or pharmaceutically acceptable compound, is administered to a patient at a dose of about 200 mg at least once a day for about two weeks to about six months, and then the dose is increased to about 400 mg at least once a day for at least 21 days.

In an aspect, the dosing protocols comprise a compound of Formula I or pharmaceutically acceptable salt thereof, for use in treating cancer, wherein the compound, or pharmaceutically acceptable compound, is administered to a patient at a dose of about 400 mg at least once a day.

In an aspect, the dosing protocols comprise a compound of Formula I or pharmaceutically acceptable salt thereof, for use in treating cancer, wherein the compound, or pharmaceutically acceptable compound, is administered to a patient at a dose of about 400 mg at least once a day for about two weeks to about six months, and then the dose is decreased to about 200 mg at least once a day for at least 21 days.

In an aspect, the dosing protocols comprise a compound of Formula I or pharmaceutically acceptable salt thereof, for use in simultaneous, separate or sequential combination with a second therapeutic in treating cancer in a patient, wherein the compound, or pharmaceutically acceptable salt thereof, is administered at a dose of between about 200 mg and about 400 mg at least once a day for at least one week.

In an aspect, the dosing protocols comprise a compound of Formula I or pharmaceutically acceptable salt thereof, for use in simultaneous, separate or sequential combination with a second therapeutic and a third therapeutic in treating cancer in a patient, wherein the second therapeutic and the third therapeutic are different and wherein the compound, or pharmaceutically acceptable salt thereof, is administered at a dose of between about 200 mg and about 400 mg at least once a day for at least one week.

In an aspect, the dosing protocols comprise a compound of Formula I or pharmaceutically acceptable salt thereof, for use in simultaneous, separate or sequential combination with a second therapeutic in treating cancer in a patient, wherein the cancer is selected from the group consisting of: breast cancer including metastatic breast cancer (mBC) and advanced breast cancer, ovarian cancer, endometrial cancer, including endometrioid endometrial cancer (EEC), prostate cancer, uterine cancer, gastric cancer, and lung cancer, the compound or pharmaceutically acceptable salt thereof is administered at a dose between about 200 mg and about 400 mg.

In an aspect, disclosed herein is a compound of Formula I or pharmaceutically acceptable salt thereof, for use in treating cancer in a patient, wherein the compound, or pharmaceutically acceptable salt thereof, is administered at a dose of about 400 mg at least once a day. In an embodiment, the breast cancer is ER+, HER2−, metastatic breast cancer. In another embodiment, the breast cancer is ER+, HER2−, advanced breast cancer.

In an aspect, the dosing protocols comprise a compound which is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1) for use in simultaneous, separate or sequential combination with pertuzumab and trastuzumab for treating ER+, HER2-negative breast cancer.

In an aspect, the dosing protocols comprise a compound which is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1) for use in simultaneous, separate or sequential combination with pertuzumab and trastuzumab for treating ER+, HER2-positive breast cancer.

In an aspect, the dosing protocols comprise a compound which is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1) for use in simultaneous, separate or sequential combination with abemaciclib for treating ER+, HER2-negative breast cancer, wherein the compound is administered at a dose of about 400 mg. In an embodiment, the compound is administered at a dose of about 200 mg. In another embodiment, the compound is administered at a dose of about 300 mg.

The protocols use the compound of Formula I or a pharmaceutically acceptable salt thereof in combination with one or more other therapeutic agents, in conjunction with surgery, or in combination with both one or more other therapeutic agents and in conjunction with surgery, in adjuvant therapy. In an embodiment, the compound of Formula I or a pharmaceutically acceptable salt thereof is administered for at least a week, prior to surgery. In an alternate embodiment, the compound of Formula I or a pharmaceutically acceptable salt thereof is administered for at least a week, after surgery. In another embodiment, the compound of Formula I or a pharmaceutically acceptable salt thereof is administered for at least a week, prior to surgery and for at least a week, after surgery. Preferably, the compound is (5R)-5-[4-[2[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In one aspect, disclosed herein is a method of treating a cancer comprising: administering a dose between about 200 mg and about 400 mg of a compound of Formula I or a pharmaceutically acceptable salt thereof to a patient in need of such treatment, at least once a day for at least one week.

Preferably, the pharmaceutically acceptable salt of the compound of Formula I is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In another aspect, disclosed herein is a method of treating cancer, the method comprising: administering a dose of about 200 mg of the compound of Formula I or a pharmaceutically acceptable salt thereof at least once a day, to a patient in need of such treatment, for about two weeks to about six months, and then increasing the dose to about 300 mg at least once a day, for at least 14 days or at least 21 days or at least 28 days. Preferably, the compound is (5R)-5-[4-2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In still another aspect, disclosed herein is a method of treating cancer, the method comprising: administering a dose of about 200 mg of the compound of Formula I or a pharmaceutically acceptable salt thereof at least once a day, to a patient in need of such treatment, for about two weeks to about six months, and then increasing the dose to about 400 mg at least once a day, for at least 14 days, or at least 21 days, or at least 28 days. Preferably, the compound is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In yet another aspect, disclosed herein is a method of treating cancer, the method comprising: administering a dose of about 400 mg of the compound of Formula I or a pharmaceutically acceptable salt thereof at least once a day, to a patient in need of such treatment, for about two weeks to about six months, and then decreasing the dose to about 200 mg at least once a day for at least 14 days, or at least 21 days, or at least 28 days. Preferably, the compound is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In a further aspect, disclosed herein is the use of a compound of Formula I or a pharmaceutically acceptable salt thereof to treat cancer, the use comprising administering about 200 mg of the compound of Formula I or a pharmaceutically acceptable salt thereof at least once a day, to a patient in need of such treatment, for about two weeks to about six months, and then increasing the dose to about 400 mg at least once a day, for at least 14 days or at least 21 days or at least 28 days. Preferably, the compound is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In a still further aspect, disclosed herein is the use of a compound of Formula I or a pharmaceutically acceptable salt thereof to treat cancer, the use comprising administering about 400 mg of the compound of Formula I or a pharmaceutically acceptable salt thereof at least once a day, to a patient in need of such treatment, for about two weeks to about six months, and then decreasing the dose to about 200 mg at least once a day for at least 14 days or at least 21 days or at least 28 days. Preferably, the compound is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In a yet still further aspect, disclosed herein is the use of the compound of Formula I or a pharmaceutically acceptable salt thereof in the manufacture of a medicament for treating cancer, wherein the medicament comprises about 200 mg to about 400 mg of the compound or salt thereof, and the medicament is administered at least once a day for at least one week. Preferably, the compound is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In an aspect, the dosing protocols comprise a compound which is 400 mg of (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1), for use in simultaneous, separate or sequential combination with 6 mg/kg of pertuzumab (Q21D) and 420 mg trastuzumab (Q21D), for treating ER+, HER2-negative breast cancer, where Q21D means every 21 days.

In an aspect, the dosing protocols comprise a compound which is 400 mg of (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1), for use in simultaneous, separate or sequential combination with 6 mg/kg of pertuzumab (Q21D) and 420 mg trastuzumab (Q21D), for treating ER+, HER2-positive breast cancer, where Q21D means every 21 days.

In an aspect, the compound of Formula I or pharmaceutically acceptable salt thereof is administered for about two weeks, to a patient having ER-positive, human epidermal growth factor receptor 2 negative (HER2-negative) early stage (stage I-III) breast cancer. Preferably, the compound is (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1).

In an aspect, disclosed herein are methods of treating a cancer comprising: administering a dose between about 200 mg and about 400 mg of a compound of Formula I or pharmaceutically acceptable salt thereof, a second therapeutic, and a third therapeutic, wherein the second therapeutic and the third therapeutic are different, to a patient in need of such treatment, at least once a day for at least one week. In all aspects, a preferred pharmaceutically acceptable salt of the compound of Formula I is the tosylate salt, i.e., the 4-methylbenezenesulfonic acid salt.

### BRIEF DESCRIPTION OF THE FIGURES

FIG. 1 is a chart showing mean (+Standard Deviation) total plasma concentration time profiles on Day 15 of a clinical trial dose escalation study following multiple oral doses of (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1) within the range of 200 mg to 1200 mg QD.

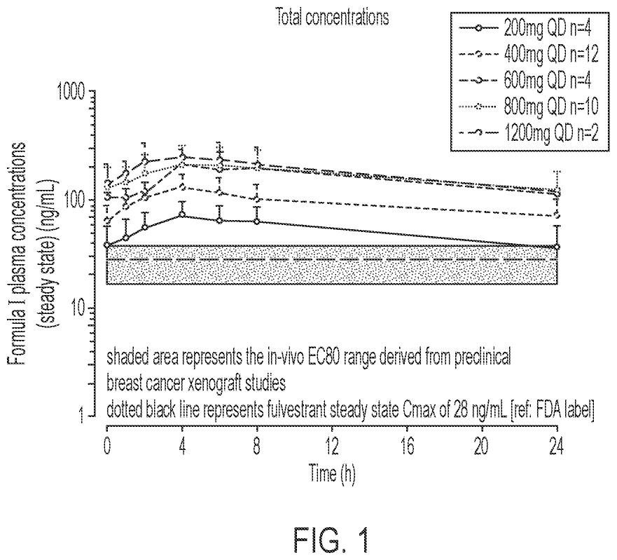

FIG. 2 is a chart showing mean (+Standard Deviation) unbound plasma concentration time profiles on Day 15 in the clinical trial dose escalation study following multiple oral doses of (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1) within the range of 200 mg to 1200 mg QD.

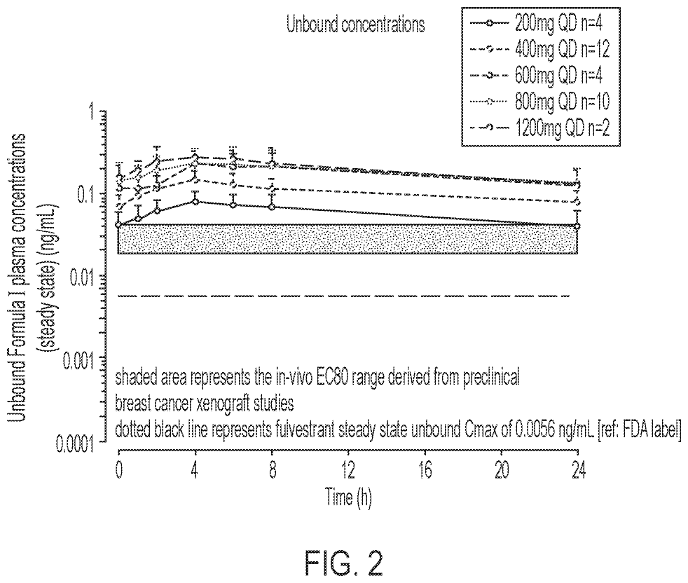

### DETAILED DESCRIPTION

### Pharmaceutically Acceptable Salts

The compound of Formula I is preferably used as the tosylate salt, which is also known in the art as the 4-methylbenzenesulfonic acid salt or the p-toluenesulfonic acid salt.

However, other pharmaceutically acceptable, acid addition salts may be utilized. Such pharmaceutically acceptable acid addition salts and methods of preparing them are known. For example, See, e.g., P. Stahl, et al., HANDBOOK OF PHARMACEUTICAL SALTS: PROPERTIES, SELECTION AND USE, (VCHA/Wiley-VCH, 2002); L. D. Bighley, S. M. Berge, D. C. Monkhouse, in “Encyclopedia of Pharmaceutical Technology’. Eds. J. Swarbrick and J.C. Boylan, Vol. 13, Marcel Dekker, Inc., New York, Basel, Hong Kong 1995, pp. 453-499; S.M. Berge, et al., “Pharmaceutical Salts”, Journal of Pharmaceutical Sciences, Vol 66, No. 1, January 1977. Specific examples of acids that could be used to prepare these other salts include methanesulfonic acid (which forms the mesylate salt), benzene sulfonic acid (which forms the besylate salt), trifluoromethane sulfonic acid (which forms the triflate salt), HCl, H₂SO₄, HNO₃, and H₃PO₄.

### Cancers

In one embodiment, the cancer is selected from the group consisting of breast cancer, including advanced breast cancer, metastatic breast cancer (mBC), ovarian cancer, endometrial cancer, including endometrioid endometrial cancer (EEC), prostate cancer, uterine cancer, gastric cancer, and lung cancer. In one embodiment, the cancer is breast cancer and/or endometrial cancer.

In one embodiment, the cancer is a cancer that is hormone receptor positive (HR-positive) such that the cancer cells express hormone receptors. Hormone receptors include both estrogen receptors and progesterone receptors. In one embodiment, the cancer is estrogen receptor positive (ER-positive). In one embodiment, the cancer expresses tyrosine kinase receptors, such as HER2. The cancer may be HER2-positive or HER2-negative.

In an embodiment, the cancers that can be treated using the compound of Formula I or pharmaceutically acceptable salts thereof are those that are HR-positive, such as ER-positive, and tyrosine kinase receptors, such as HER2-positive or HER2-negative

In an embodiment, the cancer is ER+, HER2− breast cancer. The breast cancer can be advanced or metastatic.

### Dosing Regimen

In an embodiment, a compound of Formula I or a pharmaceutically acceptable salt is administered to a patient in need of such treatment, at doses of about 200 mg to about 1200 mg. Doses of about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 150 mg, about 1000 mg, about 1050 mg, about 1100 mg, about 1150 mg, or about 1200 mg may be administered. The daily maximum dose, i.e., the maximum dose in a 24 hour period, is no more than about 1200 mg. In some embodiments, the dose is about 200 to about 1000 mg or about 200 to about 800 mg, or about 200 mg to about 600 mg, or about 200 mg to about 400 mg. Preferably, the dose is about 200 mg to about 400 mg. In a preferred embodiment, the dose is 400 mg. Preferably, the compound of Formula I is (5R)-)-5-[4-[2-[3-(fluoromethyl)azetidin-1-yl]ethoxy]phenyl]-8-(trifluoromethyl)-5H-chromeno[4,3-c]quinolin-2- ol, 4-methylbenzenesulfonic acid (1/1). Preferably the dose is administered at least once a day for at least one week. The dose can be administered more than once a day.

### Prior Therapies

In an embodiment, the patient in need of therapy has previously had endocrine therapy, where endocrine therapy is hormone therapy that is used to treat cancer. In some embodiments, the patient has been diagnosed as having sensitivity to endocrine therapy. In some embodiments, the patient has not had cyclin-dependent kinases (CDK4/6) inhibitor-containing therapy.

In another embodiment, disclosed herein is a method wherein the patient in need of therapy has had no more than one prior therapy. In yet another embodiment disclosed herein is a method wherein the patient has had no more than two prior therapies. In yet another embodiment disclosed herein is a method wherein the patient has had no more than three prior therapies. In yet another embodiment disclosed herein is a method wherein the patient has had no more than four prior therapies. In some embodiments, the patient has had eight prior therapies or more.

As used herein, “prior therapy” refers to treatment that was previously administered or used in an effort to treat the cancer. Administering a single medication or administering two or more medications as part of an adjuvant therapy, are examples of prior therapy. Surgery is also an example of prior therapy. Endocrine therapy and aromatase inhibitor therapy are examples of prior therapy, as is treatment with a platinum based chemotherapeutic drug, a CDK4/6 inhibitor, or treatment with fulvestrant. Treating a patient with a medication, followed by surgery, is an example of two prior therapies.

The compound of Formula I and pharmaceutically acceptable salts thereof are administered to a patient that has experienced or exhibited at least one symptom of the cancer to be treated. In some embodiments, patient has not had CDK4/6 inhibitor-containing therapy. In another embodiment, the patient has previously had endocrine therapy.

In still another embodiment disclosed herein is a method wherein the patient has previously had endocrine therapy.

In another embodiment, disclosed herein is a method wherein the patient in need of therapy has been identified or diagnosed as having EEC. In another embodiment disclosed herein is a method wherein the EEC is ER-positive. In another embodiment, the EEC has not been treated by platinum therapy. In another embodiment the EEC has been treated by platinum therapy. In another embodiment disclosed herein is a method wherein the EEC has progressed after being treated by platinum therapy. In another embodiment disclosed herein is a method wherein the EEC has not been treated by fulvestrant or aromatase inhibitor therapy.

### Monotherapy

In another embodiment disclosed herein is a method of treating a cancer including administering a dose between about 200 mg to about 800 mg or about 200 mg to about 400 mg of a compound of Formula I or a pharmaceutically acceptable salt thereof to a patient in need thereof. In yet another embodiment disclosed herein is a method of treating a cancer including administering a dose between about 200 mg to about 800 mg or about 200 mg to about 400 mg of a compound of Formula I or a pharmaceutically acceptable salt thereof to a patient in need thereof at least once a day for at least one week.

In another embodiment disclosed herein is a method comprising administering a dose of about 200 mg, about 300 mg, about 400 mg or about 800 mg of a compound of Formula I or a pharmaceutically acceptable salt thereof to a patient in need thereof. In an embodiment, the method comprises administering a dose of about 200 mg. In an alternate embodiment, the method comprises administering a dose of about 400 mg.

In another embodiment disclosed herein is a method wherein the dose of a compound of Formula I or a pharmaceutically acceptable salt thereof is about 200 mg to a patient in need thereof. In another embodiment disclosed herein is a method further comprising the steps of administering a dose of a compound of Formula I or a pharmaceutically acceptable salt thereof of about 200 mg to the patient at least once a day; followed by administering an increased dose of about 300 mg to about 400 mg to the patient at least once a day. In another embodiment disclosed herein is a method wherein the step of administering the dose of about 200 mg occurs prior to surgery. In another embodiment disclosed herein is a method wherein the step of administering the increased dose occurs following surgery. In another embodiment the disclosure provides a method wherein the step of administering the increased dose occurs daily for at least 3 months up to the end of the patient's life.

In another embodiment disclosed herein is a method wherein the dose of a compound of Formula I or a pharmaceutically acceptable salt thereof is about 400 mg to a patient in need thereof. In another embodiment disclosed herein is a method further comprising the steps of administering a dose of about 400 mg to the patient at least once a day; followed by administering a decreased dose of about 200 mg to about 300 mg to the patient at least once a day. In another embodiment disclosed herein is a method wherein the step of administering the dose of about 400 mg occurs prior to surgery. In an embodiment, the patient is scheduled to have surgery to treat the EEC, and the dose of the compound of Formula I or a pharmaceutically acceptable salt thereof is about 400 mg, and the dose is administered at least once a day, for at least one week, prior to the surgery. In a further embodiment, the 400 mg dose is administered at least once a day for about two weeks, prior to surgery. In another embodiment disclosed herein is a method wherein the step of administering the decreased dose occurs following surgery. In an embodiment, the dose of the compound of Formula I or pharmaceutically acceptable salt thereof is decreased to about 200 mg at least once a day, after the surgery. In another embodiment, the dose of the compound of Formula I or pharmaceutically acceptable salt thereof is decreased to about 300 mg at least once a day, after the surgery. In another embodiment disclosed herein is a method wherein the step of administering the decreased dose occurs daily for at least one day, at least one week, at least two weeks, at least four weeks, at least 2 months, or at least 3 months, or up to the end of the patient's life.

In another embodiment, the compound of Formula I or pharmaceutically acceptable salt thereof is administered at a dose of about 200 mg to the patient at least once a day; for at least one week, followed by administering an increased dose of about 300 mg to about 400 mg to the patient at least once a day for at least one week. When the patient is scheduled to have surgery to treat the EEC, a dose of about 200 mg is administered at least once a day, for at least one week prior to surgery. In some embodiments, the 200 mg dose is administered at least once a day for about two weeks, prior to surgery. In further embodiments, the dose of the compound of Formula I or pharmaceutically acceptable salt thereof is increased to about 300 mg at least once a day, after the surgery or alternatively the dose of the compound of Formula I or pharmaceutically acceptable salt thereof is increased to about 400 mg at least once a day, after the surgery. If desired, the step of administering the increased dose occurs daily for at least one week.

In an embodiment, disclosed herein is a method wherein the patient in need of therapy is scheduled to have surgery to treat cancer, and a dose of a compound of Formula I or a pharmaceutically acceptable salt thereof of about 400 mg is administered at least once a day, for at least one week, prior to the surgery. In a further embodiment, the cancer is metastatic breast cancer. In another embodiment, the cancer is advanced breast cancer. In another embodiment disclosed herein is a method wherein the patient in need of therapy is scheduled to have surgery to treat the EEC, and a dose of a compound of Formula I or a pharmaceutically acceptable salt thereof of about 400 mg is administered at least once a day, for at least one week, prior to the surgery.

In another embodiment disclosed herein is a method wherein the patient in need of therapy has been identified or diagnosed as having a cancer selected from the group consisting of breast cancer, including mBC, advanced breast cancer, ovarian cancer, endometrial cancer, including EEC, prostate cancer, uterine cancer, gastric cancer, and lung cancer. In another embodiment disclosed herein is a method wherein the breast cancer is ER-positive. In another embodiment disclosed herein is a method wherein the breast cancer is HER2-negative. In another embodiment disclosed herein is a method wherein the breast cancer is HER2-positive. In a preferred embodiment, the cancer is ER+ and HER2−. Still more preferably, in one embodiment, the cancer is ER+and HER2− breast cancer.

In another embodiment disclosed herein is a method wherein the patient in need of therapy has been identified or diagnosed as having mBC. In another embodiment disclosed herein is a method wherein the mBC is HER2-negative. In another embodiment disclosed herein is a method wherein the mBC is HER2-positive. In another embodiment disclosed herein is a method wherein the mBC has not been treated or is de novo, wherein de novo means starting from the beginning or starting anew.

In another embodiment disclosed herein is a method wherein the patient in need of therapy has been identified or diagnosed as having advanced breast cancer. In another embodiment disclosed herein is a method wherein the advanced breast cancer is HER2-negative. In another embodiment disclosed herein is a method wherein the advanced breast cancer is HER2-positive. In another embodiment disclosed herein is a method wherein the advanced breast cancer has not been treated or is de novo, wherein de novo means starting from the beginning or starting anew.

In another embodiment, disclosed herein is a method wherein the patient in need of therapy has a breast cancer that is ER-positive (ER+) and HER2-positive (HER2+). In another embodiment, disclosed herein is a method wherein the patient in need of therapy has breast cancer and the breast cancer is locally advanced, unresectable, or metastatic.

In another embodiment, disclosed herein is a method wherein the patient in need of therapy has a breast cancer that is ER-positive (ER+) and HER2-negative (HER2−). In another embodiment, disclosed herein is a method wherein the patient in need of therapy has breast cancer and the breast cancer is locally advanced, unresectable, or metastatic..

In another embodiment, patients have received induction taxane chemotherapy combined with trastuzumab and pertuzumab as the first-line treatment regimen. In another embodiment, patients are considered appropriate for continued treatment with trastuzumab and pertuzumab. In another embodiment, patients have not progressed on the first line treatment regimen. In another embodiment, patients have progressed on the first line treatment regimen. In another embodiment, patients have not received more than one HER2− directed regimen or any endocrine therapy for advanced disease, or any prior CDK4/6 inhibitor therapy.

In another embodiment, patients have Left Ventricular Ejection Fraction (LVEF) of 50% or higher at baseline as determined by echocardiography or multigated acquisition scanning. In another embodiment, patients do not have Left Ventricular Ejection Fraction (LVEF) of 50% or higher at baseline as determined by echocardiography or multigated acquisition scanning.

### Adjuvant Therapy

Disclosed herein is a method of treating cancer, the method further comprising administering a second therapeutic agent. In an embodiment the method of treating cancer comprises administering a dose between about 200 mg and about 400 mg of a compound of Formula I or a pharmaceutically acceptable salt thereof in combination with a second therapeutic agent, to a patient in need of therapy at least once a day. In an embodiment, the compound of Formula I or a pharmaceutically acceptable salt thereof is administered for at least one week. In another embodiment disclosed herein is a method wherein the dose of a compound of Formula I or a pharmaceutically acceptable salt thereof is about 200 mg, about 300 mg, or about 400 mg. In another embodiment disclosed herein is a method wherein the dose is about 200 mg. In another embodiment disclosed herein is a method wherein the dose is about 300 mg. In another embodiment disclosed herein is a method wherein the dose is about 400 mg.

In another embodiment disclosed is a method of treating a cancer including administering a dose between about 200 mg and about 400 mg of a compound of Formula I or a pharmaceutically acceptable salt thereof in combination with the second therapeutic agent and a third therapeutic agent to a patient in need thereof at least once a day, for at least one week. In another embodiment disclosed herein is a method wherein the dose is about 200 mg, about 300 mg, or about 400 mg. In an embodiment, the dose of the compound of Formula I is about 400 mg.

In all aspects and embodiments disclosed herein, the second therapeutic agent, is administered simultaneously, separately, or sequentially with the compound of Formula I or the pharmaceutically acceptable salt thereof. The third therapeutic agent is administered simultaneously, separately or sequentially with the second therapeutic agent and/or the compound of Formula I or the pharmaceutically acceptable salt thereof.

In some embodiments, the second therapeutic agent is selected from the group consisting of abemaciclib, an aromatase inhibitor, everolimus, alpelisib, trastuzumab, and pertuzumab. In some embodiments, the aromatase inhibitor is selected from the group consisting of: anastrozole, exemestane, and letrozole. In some embodiments, the second therapeutic agent is abemaciclib. In an alternate embodiment, the second therapeutic agent is trastuzumab. In a preferred embodiment, the dose of the compound of Formula I or pharmaceutically acceptable salt thereof is 400 mg, and the second therapeutic agent is abemaciclib.

In some embodiments, ER+, HER2− advanced breast cancer is treated with about 400 mg of the compound of Formula I or pharmaceutically acceptable salt thereof, and the second therapeutic agent is abemaciclib.

In other embodiments, ER+, HER2− metastatic breast cancer is treated with about 400 mg of the compound of Formula I or pharmaceutically acceptable salt thereof, and the second therapeutic agent is abemaciclib.

In some embodiments, the third therapeutic agent is selected from the group consisting of: an aromatase inhibitor, everolimus, alpelisib, trastuzumab, and pertuzumab. The third therapeutic agent differs from the second therapeutic agent. In some embodiments, the aromatase inhibitor is selected from the group consisting of: anastrozole, exemestane, and letrozole. In some embodiments, the third therapeutic agent is selected from the group consisting of an aromatase inhibitor and trastuzumab. In some embodiments, the aromatase inhibitor is selected from the group consisting of: anastrozole, exemestane, and letrozole. In an embodiment, the third therapeutic agent is an antidiarrheal agent. In an embodiment, the third therapeutic agent is pertuzumab. In other embodiments, the second therapeutic agent is trastuzumab and the third therapeutic agent is pertuzumab.

Examples of antidiarrheal agents include, but are not limited to antidiarrheal agent is selected from the group consisting of Lactobacillus acidophilus, atropine/diphenoxylate, atropine/difenoxin, loperamide, bismuth subsalicylate, loperamide, Saccharomyces boulardii Lyoactobacillus acidophilus/Lactobacillus bulgaricus, Lactobacillus rhamnosus gg, and crofelemer. In an embodiment, the antidiarrheal agent is loperamide.

In another embodiment disclosed herein is a method wherein the dose a compound of Formula I or a pharmaceutically acceptable salt thereof is about 200 mg. In another embodiment disclosed herein is a method further comprising the steps of administering a dose of about 200 mg to the patient at least once a day; followed by administering an increased dose of about 300 mg to about 400 mg to the patient at least once a day. In another embodiment disclosed herein is a method wherein the step of administering the dose of about 200 mg occurs prior to surgery. In another embodiment disclosed herein is a method wherein the step of administering the increased dose occurs following surgery. In another embodiment disclosed herein is a method wherein the step of administering the increased dose occurs daily for at least 3 months up to the end of the patient's life.

In another embodiment disclosed herein is a method wherein the dose a compound of Formula I or a pharmaceutically acceptable salt thereof is about 400 mg. In another embodiment disclosed herein is a method further comprising the steps of administering a dose of a compound of Formula I or a pharmaceutically acceptable salt thereof of about 400 mg to the patient at least once a day; followed by administering a decreased dose of about 200 mg to about 300 mg to the patient at least once a day. In another embodiment disclosed herein is a method wherein the step of administering the dose of about 400 mg occurs prior to surgery. In another embodiment disclosed herein is a method wherein the step of administering the decreased dose occurs following surgery. In another embodiment disclosed herein is a method wherein the step of administering the decreased dose occurs daily for at least 3 months up to the end of the patient's life.

In some embodiments, the compound of Formula I or pharmaceutically acceptable salt thereof as described herein may be utilized in combination with one or more other therapies to treat a relevant disease, disorder, or condition. In some embodiments, dosing of the compound of Formula I or pharmaceutically acceptable salt thereof is altered when utilized in adjuvant therapy as compared with when administered as monotherapy. Alternatively or additionally, in some embodiments, a therapy that is administered in combination with the compound of Formula I or pharmaceutically acceptable salt thereof as described herein is administered according to a regimen or protocol that differs from its regimen or protocol when administered alone or in combination with one or more therapies other than the compound of Formula I. In some embodiments, compositions which comprise an additional therapeutic agent, that additional therapeutic agent and a provided compound may act synergistically. In some embodiments, the compound of Formula I or pharmaceutically acceptable salt thereof may act synergistically in combination with a second therapeutic agent, or a pharmaceutically acceptable salt thereof. In some embodiments, one or both therapies utilized in a combination regimen is administered at a lower level or less frequently than when it is utilized as monotherapy.

In some embodiments, the compound of Formula I or pharmaceutically acceptable salt thereof may be administered to a patient in need of treatment at a dose between about 200 mg and about 400 mg in combination with one or more other therapeutic agents including in combination with a second therapeutic agent, or a pharmaceutically acceptable salt thereof. In some embodiments, the dose is about 200 mg or about 400 mg. In some embodiments, the dose is 200 mg. In some embodiments, additional steps include administering a dose of about 200 mg to the patient at least once a day; followed by administering an increased dose of about 300 mg to about 400 mg to the patient at least once a day. In some embodiments, the dose is 400 mg. In some embodiments, additional steps include administering a dose of about 400 mg to the patient at least once a day; followed by administering a decreased dose of about 200 mg to about 300 mg to the patient at least once a day.

In some embodiments, the compound of Formula I or pharmaceutically acceptable salt thereof may be administered at a dose between about 200 mg and about 400 mg in combination with one or more other therapeutic agents including in combination with a second therapeutic agent, or a pharmaceutically acceptable salt thereof, and a third therapeutic agent, or a pharmaceutically acceptable salt thereof.

In another embodiment, a different or additional compound is also administered. Examples of different or additional compounds include, but are not limited to additional anti-cancer drugs including other inhibitors along the MAPK pathway including inhibitors along the RAS/RAF pathway including tyrosine kinase inhibitors such as Ruxolitinib, Ponatinib, Erlotinib, Alectinib, Osimertinib, Afatinib, Bosutinib, Axitinib, Ceritinib, Acalabrutinib, Sunitinib, Lenvatinib, Brigatinib, Imatinib, Neratinib, Lapatinib, Crizotinib, Cabozantinib, Ibrutinib, Dasatinib, Gefitinib, or Binimetinib; inhibitors along the MEK1/2 cascade including inhibitors along the ERK pathway including ulixertinib, MK-8353, LTT-462, ASTX029 and JSI-1187.

BRAF kinase inhibitors such as Trametinib, Vemurafenib, Dabrafenib, Sorafenib, or Regorafenib, PARP inhibitors such as Olaparib, Rucaparib, or Niraparib, and monoclonal antibodies such as (Cetuximab) Erbitux. In some embodiments, additional anti-cancer drugs include polyfunctional alkylating agents such as Nitrosoureas, Mustards (Nitrogen Mustards), Methanesulphonates (Busulphan), or Ethylenimines; non-polyfunctional Alkylating Drugs such as Procarbazine (Matulane), Dacarbazine (DTIC), Altretamine (Hexalen), or Cisplatin (Platinol); Antimetabolites such as antifolic acid compounds (Methotrexate) or Amino acid Antagonists (Azaserine); Purine antagonists such as Mercaptopurine (6-MP), Thioguanine (6-TG), Fludarabine Phosphate, Cladribine (Leustatin), or Pentostatin (Nipent); pyrimidine antagonists such as Fluorouracil (5-FU), Cytarabine (ARA-C), or Azacitidine; Plant alkaloids such as Vinblastine (Velban), Vincristine (Oncovin), Etoposide (VP-16,VePe-sid), Teniposide (Vumon), Topotecan (Hycamtin), Irinotecan (Camptosar), Paclitaxel (Taxol), or Docetaxel (Taxotere); Antibiotics such as Anthracyclines, Doxorubicin (Adriamycin, Rubex, Doxil), Daunorubicin (DaunoXome), Dactinomycin (Cosmegen), Idarubincin (Idamycin), Plicamycin (Mithramycin), Mitomycin (Mutamycin), or Bleomycin (Blenoxane); Hormonal agents such as Tamoxifen (Nolvadex), Flutamide (Eulexin), Gonadotropin-Releasing Hormone Agonists, (Leuprolide and Goserelin (Zoladex)), Aromatase Inhibitors, Aminoglutethimide, or Anastrozole (Arimidex); or other anticancer drugs such as Amsacrine, Hydroxyurea (Hydrea), Asparaginase (El-spar), Mitoxantrone (Novantrone), Mitotane, Retinoic Acid Derivatives, Bone Marrow Growth Factors, or Amifostine.

Examples of different or additional compounds include, but are not limited to antidiarrheal drugs such as Intestinex (Lactobacillus acidophilus), Lonox (atropine/diphenoxylate), Motofen (Pro) (atropine/difenoxin), Acidophilus (Lactobacillus acidophilus), Florajen (Lactobacillus acidophilus), Imodium A-D (loperamide), Kaopectate (bismuth sub salicylate), Imotil (loperamide), Pink Bismuth (bismuth sub salicylate), Pepto-Bismol (bismuth subsalicylate), Lomotil (Pro) (atropine/diphenoxylate), Diamode (loperamide), Imodium (Pro) (loperamide), Florastor (saccharomyces boulardii lyo), Kapectolin (New Formula) (bismuth subsalicylate), Florastor Kids (saccharomyces boulardii lyo), Bacid (LAC) (Lactobacillus acidophilus), BD Lactinex (Lactobacillus acidophilus/Lactobacillus bulgaricus), Bismarex (bismuth sub salicylate), Bismatrol (bismuth sub salicylate), Bismatrol Maximum Strength (bismuth subsalicylate), Culturelle Digestive Health (Lactobacillus rhamnosus gg), Culturelle Health and Wellness (Lactobacillus rhamnosus gg), Dofus (Lactobacillus acidophilus), Flora-Q (Lactobacillus acidophilus), Floranex (Lactobacillus acidophilus/Lactobacillus bulgaricus), Fulyzaq (Pro) (Crofelemer), Kao-Paverin (loperamide), Kola-Pectin DS (bismuth subsalicylate), Lomocot (atropine/diphenoxylate), Mytesi (Pro) (Crofelemer), Novaflor (Lactobacillus acidophilus), Peptic Relief (bismuth subsalicylate), Percy Medicine (bismuth subsalicylate), Risa-Bid (Lactobacillus acidophilus), RisaQuad (Lactobacillus acidophilus), Soothe Caplets (bismuth sub salicylate), or Superdophilus (Lactobacillus acidophilus). In some embodiments, the use includes administration of an antidiarrheal agent.

In some aspects, a compound of Formula I or pharmaceutically acceptable salt thereof with or without a second therapeutic agent or a pharmaceutically acceptable salt thereof, or a second therapeutic agent and a third therapeutic agent, or a pharmaceutically acceptable salt thereof are formulated as pharmaceutical compositions administered by any route which makes each of these compounds bioavailable. The route of administration may be varied in any way, limited by the physical properties of the drugs and the convenience of the patient and the caregiver.

In some aspects, the compound of Formula I or pharmaceutically acceptable salt thereof with or without a second therapeutic agent or a pharmaceutically acceptable salt thereof, or a second therapeutic agent and a third therapeutic agent, or a pharmaceutically acceptable salt thereof is administered orally. Alternatively, the compound of Formula I or pharmaceutically acceptable salt thereof with or without a second therapeutic agent or a pharmaceutically acceptable salt thereof, or a second therapeutic agent and a third therapeutic agent, or a pharmaceutically acceptable salt thereof, is formulated for parenteral administration, such as intravenous (IV) or subcutaneous administration. In some embodiments, one of the compound of Formula I, the second therapeutic agent, the third therapeutic agent, or a pharmaceutically acceptable salt thereof, is formulated for oral administration. In some embodiments, one of the compound of Formula I, the second therapeutic agent, the third therapeutic agent, or a pharmaceutically acceptable salt thereof, is formulated for parenteral administration, such as IV administration. In some embodiments, one of the compound of Formula I, the second therapeutic agent, the third therapeutic agent, or a pharmaceutically acceptable salt thereof, is formulated for IV administration. Such pharmaceutical compositions and processes for preparing the same are well known in the art. (See, e.g., Remington: The Science and Practice of Pharmacy, L. V. Allen, Editor, 22^{nd} Edition, Pharmaceutical Press, 2012).

In some aspects, the present disclosure relates to a compound of Formula I or pharmaceutically acceptable salt thereof with a second therapeutic agent or a pharmaceutically acceptable salt thereof, or a second therapeutic agent and a third therapeutic agent, or a pharmaceutically acceptable salt thereof for use in simultaneous, separate, or sequential combination in the treatment of breast cancer including mBC, ovarian cancer, endometrial cancer, including EEC, prostate cancer, uterine cancer, gastric cancer, and lung cancer.

In an aspect, disclosed herein is a method of treating a cancer comprising: administering a dose between about 200 mg and about 400 mg of a compound of Formula I:

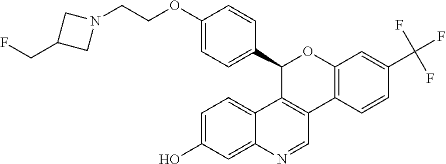 [SMILES: Oc1ccc2c3c(cnc2c1)-c1ccc(C(F)(F)F)cc1O[C@@H]3c1ccc(OCCN2CC(CF)C2)cc1]

or pharmaceutically acceptable salt thereof, a second therapeutic, and a third therapeutic, wherein the second therapeutic and the third therapeutic are different, to a patient in need of such treatment, at least once a day for at least one week. In some embodiments, the dose of the compound of Formula I is about 400 mg at least once a day. In other embodiments, the dose of the compound of Formula I is about 300 mg at least once a day. In still other embodiments, the dose of the compound of Forumula I is about 200 mg at least once a day. May different pharmaceutically acceptable salts of the compound of Formula I may be used. A preferred pharmaceutically acceptable salt is the 4-methylbenzenesulfonic acid salt.

In one preferred embodiment, the second therapeutic is trastuzumab. The trastuzumab may be administered at a dose of about 8 mg/kg at least once a day. Alternatively, the trastuzumab may be administered at a dose of about 6 mg/kg at least one a day. Alternatively, the trastuzumab may be administered at a dose of about 4 mg/kg at least one a day. In an embodiment, an initial dose of about 8 mg/kg is administered, and about 24 hours later, a dose of about 4 mg/kg is administered.

In one preferred embodiment, the third therapeutic is pertuzumab. The pertuzumab may be administered at a dose of about 840 mg. Alternatively, the pertuzumab may be administered at a dose of about 420 mg. In an embodiment, an initial dose of about 840 mg of pertuzumab is administered, and about 24 hours later, a dose of about 420 mg is administered.

In an embodiment the cancer is selected from the group consisting of breast cancer, ovarian cancer, endometrial cancer, prostate cancer, uterine cancer, gastric cancer, and lung cancer. In some embodiments, the cancer is breast cancer, and the breast cancer is advanced breast cancer, or metastatic breast cancer (mBC), and the endometrial cancer is endometrioid endometrial cancer (EEC). In certain embodiments, the cancer is HR-positive. The HR-positive cancer can be ER-positive and HER2-negative. Alternatively, the HR-positive cancer can be ER-positive and HER2-positive.

When the cancer is breast cancer, the breast cancer can be locally advanced, unresectable, or metastatic. In an embodiment, the breast cancer is metastatic breast cancer (mBC). In an embodiment, the breast cancer is advanced breast cancer. In some embodiments, the methods disclosed herein are used to treat metastatic breast cancer that has not been previously treated. In an embodiment, the advanced breast cancer has not been previously treated.

In some embodiments the methods disclosed herein are used to treat a patient that has received induction taxane chemotherapy combined with trastuzumab and pertuzumab as the first-line treatment regimen.

In some embodiments the methods disclosed herein are used to treat a patient that has not received more than one HER2− directed regimen or any endocrine therapy for advanced disease or any prior CDK4/6 inhibitor therapy.

In some embodiments the methods disclosed herein are used to treat a patient that has Left Ventricular Ejection Fraction (LVEF) of 50% or higher at baseline as determined by echocardiography or multigated acquisition scanning.

In some embodiments the methods disclosed herein are used to treat a patient that does not have Left Ventricular Ejection Fraction (LVEF) of 50% or higher at baseline as determined by echocardiography or multigated acquisition scanning.

In another embodiment, the method of treatment including the compound of Formula I, or a pharmaceutically acceptable salt thereof, trastuzumab, and pertuzumab, wherein pertuzumab is withheld or discontinued if trastuzumab is withheld or discontinued.

The disclosed SERDs that are described herein provide inhibition of ER-mediated transcription that will be useful in treating cancers such as breast cancer including mBC, ovarian cancer, endometrial cancer, including EEC, prostate cancer, uterine cancer, gastric cancer, and lung cancer as well as mutations due to emerging resistance. These SERDs can be used either as single agents or in combination with other classes of drugs including selective estrogen receptor modulators (SERMs), aromatase inhibitors, CDK4 inhibitors, CDK6 inhibitors, PI3K inhibitors, and mammalian target of rapamycin (mTOR) inhibitors to treat HR-positive cancers such as breast cancer, including advanced breast cancer, mBC, ovarian cancer, endometrial cancer, including EEC, prostate cancer, uterine cancer, gastric cancer, and lung cancer.

### Definitions

As used herein, the term “cancer” refers to or describes the physiological condition in patients that is typically characterized by unregulated cell proliferation. Included in this definition are benign and malignant cancers.

As used herein, the term “primary tumor” or “primary cancer” refer to the original cancer and not a metastatic lesion located in another tissue, organ, or location in the subject's body.

The term “polymorph,” as used herein, refers to crystals of the same compound having different physical properties as a result of the order of the molecules in the crystal lattice. Different polymorphs of a single compound have one or more different chemical, physical, mechanical, electrical, thermodynamic, and/or biological properties from each other. Differences in physical properties exhibited by polymorphs can affect pharmaceutical parameters such as storage stability, compressibility, density (important in composition and product manufacturing), dissolution rates (an important factor in determining bio-availability), solubility, melting point, chemical stability, physical stability, powder flowability, water sorption, compaction, and particle morphology. Differences in stability can result from changes in chemical reactivity (e.g., differential oxidation, such that a dosage form discolors more rapidly when comprised of one polymorph than when comprised of another polymorph) or mechanical changes (e.g., crystal changes on storage as a kinetically favored polymorph converts to a thermodynamically more stable polymorph) or both (e.g., one polymorph is more hygroscopic than the other). As a result of solubility/dissolution differences, some transitions affect potency and/or toxicity. In addition, the physical properties of the crystal may be important in processing; for example, one polymorph might be more likely to form solvates or might be difficult to filter and wash free of impurities (i.e., particle shape and size distribution might be different between one polymorph relative to the other). “Polymorph”, as used herein, does not include amorphous forms of the compound of Formula I. As used herein, “amorphous” refers to a noncrystalline form of a compound which can be a solid state form of the compound of Formula I or pharmaceutically acceptable salt thereof or a solubilized form of the compound of Formula I. For example, “amorphous” refers to a compound (e.g., a solid form of the compound) without a regularly repeating arrangement of molecules or external face planes.

The term “anhydrous,” as used herein, refers to a crystal form of the compound of Formula I or pharmaceutically acceptable salt thereof that has 1% or less by weight water. For example, 0.5% or less, 0.25% or less, or 0.1% or less by weight water.

The term “solvate” as used herein refers to a crystalline form of the compound of Formula I, such as a polymorph form of the compound of Formula I, where the crystal lattice comprises one or more solvents of crystallization.

“Purity,” when used in reference to a composition including a polymorph of the compound of Formula I, refers to the percentage of one specific polymorph form relative to another polymorph form or an amorphous form of the compound of Formula I or pharmaceutically acceptable salt thereof in the referenced composition. For example, a composition comprising polymorph Form 1 having a purity of 90% would comprise 90 weight parts Form 1 and 10 weight parts of other polymorph and/or amorphous forms of the compound of Formula I.

As used herein, a compound of Formula I or pharmaceutically acceptable salt thereof or composition is “substantially free of” one or more other components compound of Formula I or pharmaceutically acceptable salt thereof or composition contains no significant amount of such other components. For example, the composition can contain less than 5%, 4%, 3%, 2%, or 1% by weight of other components. Such components can include starting materials, residual solvents, or any other impurities that can result from the preparation of and/or isolation of Formula I s and compositions provided herein. In some aspects, a polymorph form provided herein is substantially free of other polymorph forms. In some aspects, a particular polymorph of the compound of Formula I or pharmaceutically acceptable salt thereof is “substantially free” of other polymorphs if the particular polymorph constitutes at least about 95% by weight of the compound of Formula I or pharmaceutically acceptable salt thereof present. In some aspects, a particular polymorph of the compound of Formula I or pharmaceutically acceptable salt thereof is “substantially free” of other polymorphs if the particular polymorph constitutes at least about 97%, about 98%, about 99%, or about 99.5% by weight of the compound of Formula I or pharmaceutically acceptable salt thereof present. In certain aspects, a particular polymorph of the compound of Formula I or pharmaceutically acceptable salt thereof is “substantially free” of water if the amount of water constitutes no more than about 2%, about 1%, or about 0.5% by weight of the polymorph.

As used herein, “substantially pure,” when used in reference to a polymorph form of the compound of Formula I, means a sample of a polymorph form of the compound of Formula I or pharmaceutically acceptable salt thereof having a purity greater than 90%, including greater than 90%, 91%, 92%, 93%, 94%, 95%, 96%, 97%, 98%, and 99%, and also including equal to about 100% of the compound of Formula I, based on the weight of the compound of Formula I. The remaining material comprises other form(s) of the compound, and/or reaction impurities and/or processing impurities arising from its preparation. For example, a polymorph form of the compound of Formula I or pharmaceutically acceptable salt thereof may be deemed substantially pure in that it has a purity greater than 90% of a polymorph form of the compound of Formula I, as measured by means that are at this time known and generally accepted in the art, where the remaining less than 10% of material comprises other form(s) of the compound of Formula I or pharmaceutically acceptable salt thereof and/or reaction impurities and/or processing impurities. The presence of reaction impurities and/or processing impurities may be determined by analytical techniques known in the art, such as, for example, chromatography, nuclear magnetic resonance spectroscopy, mass spectrometry, or infrared spectroscopy.

To provide a more concise description, some of the quantitative expressions herein are recited as a range from about amount X to about amount Y. It is understood that when a range is recited, the range is not limited to the recited upper and lower bounds, but rather includes the full range from about amount X through about amount Y, or any range therein.

The term “pharmaceutically acceptable carrier” or “pharmaceutically acceptable excipient” includes any and all solvents, co-solvents, complexing agents, dispersion media, coatings, antibacterial and antifungal agents, isotonic and absorption delaying agents, and the like, which are not biologically or otherwise undesirable. The use of such media and agents for pharmaceutically active substances is well-known in the art. Except insofar as any conventional media or agent is incompatible with the active ingredient, its use in the therapeutic compositions provided herein is contemplated. Supplementary active ingredients can also be incorporated into the compositions. In addition, various excipients, such as are commonly used in the art, can be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (2010); Goodman and Gilman's: The Pharmacological Basis of Therapeutics, 12th Ed., The McGraw-Hill Companies.

As used herein, the term “patient,” refers to any animal, including mammals such as humans. In some embodiments, the patient is a human.

In some embodiments, the patient has experienced and/or exhibited at least one symptom of the disease or disorder to be treated and/or prevented. In some embodiments, the patient has been identified or diagnosed as having a cancer such as breast cancer, including mBC, ovarian cancer, endometrial cancer, prostate cancer, uterine cancer, gastric cancer, and lung cancer. In some embodiments, the patient does not have bilateral invasive breast cancer.

In some embodiments, the patient has had prior therapy for an invasive or non-invasive breast cancer. In some embodiments, the patient has had no more than one prior therapy. In some embodiments, the patient has had no more than two prior therapies.

In some embodiments, the patient has previously had endocrine therapy. In some embodiments, the patient has been diagnosed as having sensitivity to endocrine therapy.

In some embodiments, the patient has not had CDK4/6 inhibitor-containing therapy.

In some embodiments, the patient has received, is scheduled to receive, or has not yet received concurrent neoadjuvant therapy with any other non-protocol anti-cancer therapy. In some embodiments, the patient has received, is scheduled to receive, or has not yet received radiotherapy to the ipsilateral chest wall for any malignancy. In some embodiments, the patient has received, is scheduled to receive, or has not yet received anti-estrogen therapy with raloxifene, tamoxifen, aromatase inhibitor, or other SERM, either for osteoporosis or prevention of breast cancer. In some embodiments, the patient has received, is scheduled to receive, or has not yet received hormone-replacement therapy within 4 weeks of the start of study treatment. In some embodiments, the patient has had major surgery within about 28 days prior to randomization to allow for post-operative healing of the surgical wound and site(s). In some embodiments, the patient is pregnant or breastfeeding. In some embodiments, the patient has certain infections such as hepatitis or tuberculosis or HIV that are not well controlled. In some embodiments, the patient has another serious medical condition.

As used herein, the terms “treat,” or “treatment” refer to therapeutic or palliative measures. Beneficial or desired clinical results include, but are not limited to, cure, alleviation, in whole or in part, of symptoms associated with a disease or disorder or condition, diminishment or reduction of the extent of disease, reversing the progression or severity of an existing symptom, disorder, condition, or disease, stopping disease progression, stabilized (i.e., not worsening) state of disease, delay, restraining, or slowing of disease progression, amelioration or palliation of the disease state (e.g., one or more symptoms of the disease), and regression or remission (whether partial or total), whether detectable or undetectable. “Treatment” can also mean prolonging survival as compared to expected survival if not receiving treatment.

The term “therapy” refers to the administration of one or more doses of an active compound or pharmaceutical agent to a patient as part of a therapeutic regimen.

In one aspect, the term “preventing” as used herein means the prevention of the onset, recurrence or spread, in whole or in part, of the disease or condition as described herein (e.g., multiple types of pain including inflammatory pain, neuropathic pain, and pain associated with cancer, surgery, and bone fracture), or a symptom thereof.

The term “progression” refers to cancer that becomes worse or spreads in the body, as defined by the National Cancer Institute (NCI Dictionary of Cancer Terms). For example, progression can include an increase in the number of cancer cells in the patient, an increase in the size of one or more tumors in the patient, an increase in tumor burden, an increase in the rate or extent of metastasis, worsening symptoms, in whole or in part, associated with the cancer, an increase in the extent of disease, and/or an acceleration of disease progression. “Progression” can also mean shortening survival as compared to expected survival if not receiving therapy. In some embodiments, progression can include detecting one or more of an increase in the percentage of blast cells, an increase in the myeloid to erythroid ratio, an increase in dysplasia (e.g., white blood cell dysplasia), an increase in the percentage of bone marrow plasma cells, and an increase in the percentage of bone marrow lymphocytes (see e.g., Sever, et al., Arch Pathol Lab Med. 2016 September; 140(9):932-49, which is incorporated by reference herein in its entirety). In some embodiments, progression can include detecting one or more of an increase in the percentage of leukocytes (e.g., polymorphonuclear leukocytes), a decrease in the number of platelets, and a decrease in hemoglobin in peripheral blood. In some embodiments, the tumor burden can be assessed using RECIST (e.g., RECIST version 1 or version 1.1). See, for example, Eisenhauer et al., Eur. J. Cancer. 2009, 45(2):228-47, which is incorporated by reference in its entirety herein. In some embodiments, the tumor burden can be assessed using PERCIST. See, for example, Wahl, et al. J. nucl. med. 2009, 50:122S-150S, which is incorporated by reference in its entirety herein.

The term “relapse” refers to the return of a disease or the signs and symptoms of a disease after a period of improvement, as defined by the National Cancer Institute (NCI Dictionary of Cancer Terms). For example, relapse can include detecting an increase in the number of cancer cells in the patient, an increase in the size of one or more tumors in the patient, an increase in tumor burden, an increase in the rate or extent of metastasis, worsening symptoms, in whole or in part, associated with the cancer, an increase in the extent of disease, and/or an acceleration of disease progression after a period of improvement. In some embodiments, relapse can include progression of the cancer after a period of improvement. In some embodiments, a period of improvement can include detecting a decrease in the number of cancer cells in a patient, a decrease in the size of one or more tumors in the patient, a decrease in tumor burden, a decrease in the rate or extent of metastasis, improving symptoms, in whole or in part, associated with the cancer, a decrease in the extent of disease, and/or a slowing of disease progression. In some embodiments, relapse can include detecting one or more of an increase in the percentage of blast cells, an increase in the myeloid to erythroid ratio, an increase in dysplasia (e.g., white blood cell dysplasia), an increase in the percentage of bone marrow plasma cells, and an increase in the percentage of bone marrow lymphocytes after a period of improvement. In some embodiments, a period of improvement can include detecting one or more of a decrease in the percentage of blast cells, a decrease in the myeloid to erythroid ratio, a decrease in dysplasia (e.g., white blood cell dysplasia), a decrease in the percentage of bone marrow plasma cells, and a decrease in the percentage of bone marrow. In some embodiments, relapse can include detecting one or more of an increase in the percentage of leukocytes (e.g., polymorphonuclear leukocytes), a decrease in the number of platelets, and a decrease in hemoglobin in peripheral blood after a period of improvement. In some embodiments, a period of improvement can include detecting one or more of a decrease in the percentage of leukocytes (e.g., polymorphonuclear leukocytes), an increase in the number of platelets, and an increase in hemoglobin in peripheral blood.

“Relapse” can also include “recurrence,” which the National Cancer institute defines as cancer that has recurred, usually after a period of time during which the cancer could not be detected. The cancer may come back to the same location in the body as the original (primary) tumor or to another location in the body (NCI Dictionary of Cancer Terms). In some embodiments, not detecting a cancer can include not detecting cancer cells in the patient, not detecting a tumor in the patient, and/or no symptoms, in whole or in part, associated with the cancer.

As used herein, the terms “intolerance” and “intolerant” can refer to the occurrence of a severe, disabling, or life-threatening adverse event that leads to unplanned hospitalization during therapy, therapy discontinuation, and/or therapy dose reduction, functional decline attributed to therapy, and/or a decrease in performance status. In some embodiments, a decrease in performance status can be assessed using the Eastern Cooperative Oncology Group (ECOG) Scale of Performance Status (see, e.g., Oken et al. Am. J. Clin. Oncol. 5:649-655 (1982), which is incorporated by reference in its entirety herein). In some embodiments, a decrease in performance status can be assessed using the Karnofsky Performance Status (see, e.g., Péus et al., BMC Med. Inform. Decis. Mak. 13: 72 (2013), which is incorporated by reference in its entirety herein). In some embodiments, the patient is a pediatric patient and the performance status is assessed by the Lansky Performance Score (see, e.g., Lansky et al., Cancer. 60(7):1651-6 (1987), which is incorporated by reference in its entirety herein).

The term “administration” or “administering” refers to a method of giving a dosage of a compound or pharmaceutical composition to a patient. The preferred method of administration can vary depending on various factors, e.g., the components of the pharmaceutical composition, the site of the disease, and the severity of the disease.

The daily dosage of the compound of Formula I, or a pharmaceutically acceptable salt, amorphous, or polymorph form thereof, a spray-dried dispersion thereof, or a pharmaceutical composition thereof as described herein may be varied over a wide range from 1.0 to 10,000 mg per adult human at least once a day, or higher, or any range therein. An effective amount of the drug is ordinarily supplied at a dosage level of from about 0.1 mg/kg to about 1000 mg/kg of body weight at least once a day, or any range therein. The range can be from about 0.5 to about 500 mg/kg of body weight at least once a day, or any range therein. The range can be from about 1.0 to about 250 mg/kg of body weight at least once a day, or any range therein. The range can be from about 0.1 to about 100 mg/kg of body weight at least once a day, or any range therein. In an example, the range may be from about 0.1 to about 50.0 mg/kg of body weight at least once a day, or any amount or range therein. In another example, the range may be from about 0.1 to about 15.0 mg/kg of body weight at least once a day, or any range therein. In yet another example, the range may be from about 0.5 to about 7.5 mg/kg of body weight at least once a day, or any amount to range therein. A pharmaceutical composition as provided herein may be administered on a regimen of 1 to 4 times at least once a day or in a single daily dose.

Optimal dosages to be administered may be determined by those skilled in the art, and will vary with the mode of administration, the strength of the preparation, the mode of administration, and the advancement of the disease condition. In addition, factors associated with the particular subject being treated, including subject age, weight, diet and time of administration, will result in the need to adjust dosages.

For oral administration, the compositions are, in some embodiments, provided in the form of tablets, pills or capsules containing, 200, 300, 400, 600, and 800 milligrams of the active ingredient for the symptomatic adjustment of the dosage to the subject to be treated.

The compound of Formula I or pharmaceutically acceptable salt thereof is administered in doses of about 200 mg to about 1200 mg, or about 200 to about 1000 mg or about 200 to about 800 mg, or about 200 mg to about 600 mg, or about 200 mg to about 400 mg. In some embodiments, the dose is about 200 mg to about 400 mg. In other embodiments, the dosage is 200 mg. In other embodiments, the dosage is 300 mg. In yet still other aspects, the dosage is 400 mg.

One skilled in the art will further recognize that human clinical trials including first-in-human, dose ranging and efficacy trials, in healthy subjects and/or those suffering from a given disorder, may be completed according to methods well known in the clinical and medical arts.

An effective amount can be determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement of or the severity of the disease or disorder; the response of the individual patient; the particular compound administered; the mode of administration; the bioavailability characteristics of the preparation administered; the dose regimen selected; the use of concomitant medication; and other relevant circumstances.

The compound of Formula I or pharmaceutically acceptable salt thereof with or without a second therapeutic agent or a pharmaceutically acceptable salt thereof, or a second therapeutic agent and a third therapeutic agent, or a pharmaceutically acceptable salt thereof may be administered orally at the particular frequency and dose determined separately.

“Adjuvant therapy” is understood to mean therapy that is given in addition to the first line therapy or after the first line therapy. The first line therapy comprises: the administration of one or more other therapeutic agents, radiation therapy, and/or surgery. The compound of Formula I may be the first line treatment or it may be used in adjuvant therapy.

“First line treatment” is the first treatment given for a disease.

By “therapeutically effective amount” or “pharmaceutically effective amount” or “effective amount” of a compound as provided herein is an amount which is sufficient to achieve the desired effect and can vary according to the nature and severity of the disease condition, and the potency of the compound of Formula I. A therapeutic effect is the relief, to some extent, of one or more of the symptoms of the disease, and can include curing a disease.

As used herein, the phrase “in combination with” refers to either the administration of a compound, or a pharmaceutically acceptable salt thereof, with a second therapeutic agent or a pharmaceutically acceptable salt thereof, or a second therapeutic agent and a third therapeutic agent, or a pharmaceutically acceptable salt thereof, either simultaneously or sequentially in any order, such as, for example, at repeated intervals as during a standard course of treatment for a single cycle or more than one cycle, such that one agent can be administered prior to, at the same time, or subsequent to the administration of the other agent, or any combination thereof, or to the administration of the compound of Formula I or pharmaceutically acceptable salt thereof with a second therapeutic agent or a pharmaceutically acceptable salt thereof, or a second therapeutic agent and a third therapeutic agent, or a pharmaceutically acceptable salt thereof, either simultaneously or sequentially in any order, such as, for example, at repeated intervals as during a standard course of treatment for a single cycle or more than one cycle, such that one agent can be administered prior to, at the same time, or subsequent to the administration of any of one or both or all of the other agents, or any combination thereof.

It is also understood that adjuvant therapy can be carried out by administering to a patient the amount or dose of the compound of Formula I or pharmaceutically acceptable salt thereof in combination with a second therapeutic agent, or a pharmaceutically acceptable salt thereof, or a second therapeutic agent, or a pharmaceutically acceptable salt thereof, and a third therapeutic agent, or a pharmaceutically acceptable salt thereof, which provides effective levels of the compound of Formula I or pharmaceutically acceptable salt thereof in combination with a second therapeutic agent, or a pharmaceutically acceptable salt thereof, or a second therapeutic agent, or a pharmaceutically acceptable salt thereof, and a third therapeutic agent, or a pharmaceutically acceptable salt thereof, in the body.

The term “metastasis” is an art known term and means the formation of an additional tumor (e.g., a solid tumor) at a site distant from a primary tumor in a patient or patient, where the additional tumor includes the same or similar cancer cells as the primary tumor.

The phrase “risk of developing a metastasis” means the risk that a patient or patient having a primary tumor will develop an additional tumor (e.g., a solid tumor) at a site distant from a primary tumor in a patient or patient over a set period of time, where the additional tumor includes the same or similar cancer cells as the primary tumor. Methods for reducing the risk of developing a metastasis in a patient or patient having a cancer are described herein.

The phrase “risk of developing additional metastases” means the risk that a patient or patient having a primary tumor and one or more additional tumors at sites distant from the primary tumor (where the one or more additional tumors include the same or similar cancer cells as the primary tumor) will develop one or more further tumors distant from the primary tumor, where the further tumors include the same or similar cancer cells as the primary tumor. Methods for reducing the risk of developing additional metastasis are described herein.

The following Examples merely serve to illustrate various aspects and embodiments of the disclosure and should not be considered as limiting the scope of the disclosure.

### EXAMPLE 1

### A Phase 3 Study of Imlunestrant vs Investigator's Choice of Endocrine Therapy, in Patients with Estrogen Receptor Positive, HER2 Negative Locally Advanced or Metastatic Breast Cancer Previously Treated with Endocrine Therapy

This is a randomized, active treatment study with 2 arms where the patient and investigator will not be blinded.

Approximately 500 patients will be randomized 1:1 to Arm A:Arm B.

### Intervention Groups and Duration of Treatment

| | Arm A | Arm B | |
|---|---|---|---|
| | LY3484356 | Investigator's Choice Endocrine Therapy | |
| Treatment | LY3484356 | Fulvestrant | Exemestane |
| Dose | 400 mg | 500 mg | 25 mg |
| Schedule | QD in 28-day | 500 mg on C1D1 and | QD in 28-day |
| | continuous | C1D15 and then on | continuous |
| | cycles | Day 1 of a 28-day cycle | cycles |
| | | starting at Cycle 2 | |
| Route | Oral | Intramuscular injection | Oral |
| | | of two 250 mg injections | |

Abbreviations: C = cycle; D = day; PO = orally; QD = once daily.

### Objectives and Endpoints

| | |
|---|---|
| Objectives | Endpoints |

Primary

| | |
|---|---|
| To compare the PFS of imlunestrant (Arm A) to the | Investigator-assessed PFS |
| standard comparator of Investigator's Choice Endocrine | |
| Therapy of either fulvestrant or exemestane (Arm B) | |

Secondary

| | |
|---|---|
| To compare OS of Arm A to Arm B | OS (key secondary endpoint) |
| To compare other efficacy objectives of Arm A to Arm B | Investigator-assessed ORR, DoR, and CBR |
| | Investigator-assessed PFS by ESR1 mutation |
| | status in plasma/ctDNA |
| | PFS by blinded Independent Review |
| | Committee (BIRC) |
| To assess the safety and tolerability of each treatment arm | Including but not limited to AEs, serious AEs, |
| | deaths, and clinical laboratory abnormalities per |
| | NCI CTCAE v5.0 |
| To evaluate the effectiveness of Arm A compared to Arm | Time to sustained worsening of the “worst pain” as |
| B based on PROs of pain using the Worst Pain NRS | measured by Worst Pain NRS |
| To assess the PK of imlunestrant | Plasma concentrations of imlunestrant |

### Patient Inclusion Criteria

Participants are eligible to be included in the study only if all of the following criteria apply:

1. Participant must be at least 18 years of age

2. Have a diagnosis of ER+, HER2− breast cancer

a. to fulfill the requirement for ER+ disease, a breast cancer must express the ER by immunohistochemistry, as defined in the relevant ASCO/CAP Guidelines (Allison et al. 2020)

b. to fulfill the requirement of HER2− disease, a breast cancer must not demonstrate, at initial diagnosis or upon subsequent biopsy, overexpression of HER2 by either immunohistochemistry (IHC) or in-situ hybridization as defined in the relevant ASCO/CAP Guidelines (Wolff et al. 2018). Although not required as a protocol procedure, a patient with a new metastatic lesion should be considered for biopsy whenever possible to reassess HER2 status prior to study entry if clinically indicated

3. Have locally advanced (not amenable to curative treatment by surgery) or metastatic disease and fulfill 1 of the following criteria:

a. relapsed with evidence of progression while on or within 12 months of completion of (neo)adjuvant AI, alone or in combination with a CDK4/6 inhibitor, with no treatment for advanced disease

b. relapsed with evidence of progression >12 months from completion of (neo)adjuvant ET, with subsequent progression on or after only 1 line of therapy with an AI, alone or in combination with a CDK4/6 inhibitor. Patients may not have received any other prior therapy (other than the aforementioned: AI, alone or in combination with a CDK4/6 inhibitor) in the advanced/metastatic setting

c. presented de novo with metastatic disease, with subsequent progression on or after only 1 line of therapy with an AI, alone or in combination with a CDK4/6 inhibitor. Patients may not have received any other prior therapy (other than the aforementioned: AI, alone or in combination with a CDK4/6 inhibitor) in the advanced/metastatic setting

4. Must be deemed appropriate for treatment with ET

5. If female, have a postmenopausal status due either surgical/natural menopause or ovarian suppression (received monthly and initiated at least 28 days prior to Cycle 1 Day 1) with a gonadotropin-releasing hormone agonist such as goserelin or leuprolide. Postmenopausal due to surgical/natural menopause requires at least 1 of the following:

a. prior bilateral oophorectomy

b. age ≥60 years

c. age <60 years, amenorrheic for at least 12 months (in the absence of chemotherapy, tamoxifen, toremifene, or ovarian suppression), and FSH and estradiol levels in the postmenopausal range

6. If female and postmenopausal status is due to ovarian suppression, participants must have a negative serum pregnancy test at baseline (within 14 days prior to enrollment) and agree to use highly effective, medically approved precautions to prevent pregnancy (see Section 10.7 Appendix 7) during the study and for 6 months following the last dose of study treatment

7. If male, must agree to use the following:

a. hormone suppression (received monthly and initiated at least 28 days prior to Cycle 1 Day 1) with a gonadotropin-releasing hormone agonist such as goserelin or leuprolide

b. highly effective methods of birth control and to not donate sperm during the study and for at least 6 months following the last dose of study drug(s), or for the duration specified in country requirements, whichever is longer

8. Have one of the following as defined by RECIST v1.1 (Eisenhauer et al. 2009; Section 10.3 Appendix 3):

measurable disease

nonmeasurable bone-only disease. Nonmeasurable bone-only disease may include any of the following:

i. blastic bone lesions

ii. lytic bone lesions without a measurable soft tissue component

iii. mixed lytic-blastic bone lesions without a measurable soft tissue component

9. Have a Performance Status of 0 or 1 on the Eastern Cooperative Oncology Group scale (Oken et al. 1982)

10. Have adequate organ function as defined in table below

| | |
|---|---|
| System | Laboratory Value |

Renal

| | |
|---|---|
| Serum creatinine or, | <1.5 × ULN OR |
| Measured creatinine clearance or, | ≥50 mL/min/1.73 m² |
| Calculated creatinine clearance | (See Section Error! Reference |
| | source not found. Appendix 5) |

Hematologic

| | |
|---|---|
| ANC | ≥1.5 × 10⁹/L |
| Platelets | ≥100 × 10⁹/L |
| Hemoglobin | ≥8 g/dL |

Note:
transfusions to increase a patient's hemoglobin level or initiation of erythropoietin or G-CSF therapy to meet enrollment criteria are not allowed in the 14 days preceding the first dose of study drug.

Hepatic

| | |
|---|---|
| Total bilirubin | ≤1.5 × ULN, patients with Gilbert's syndrome |
| | with a total bilirubin ≤3.0 times ULN and direct |
| | bilirubin within normal limits are permitted |
| ALT and AST | ≤3 × ULN |

Abbreviations: ALT = alanine aminotransferase; ANC = absolute neutrophil count; AST = aspartate aminotransferase; G-CSF = granulocyte colony stimulating factor; ULN = upper limit of normal.

11. Have discontinued previous therapies for cancer prior to receiving study drug, and recovered from the acute effects of therapy to at least Grade 1, except for residual alopecia and peripheral neuropathy, with the following therapy washout periods required prior to receiving study drug:

a. for myelosuppressive agents (for example, CDK4/6 inhibitors): at least 21 days

b. for nonmyelosuppressive agents (for example, Endocrine Therapy): 7 days or 5 half-lives, whichever is shorter

c. for investigational agents: 28 days or 5 half-lives, whichever is shorter

12. Patients must be able to swallow capsules/tablets

13. Are willing to participate for the duration of the study and to follow study procedures

14. Capable of giving signed informed consent as described in Appendix 1, which includes compliance with the requirements and restrictions listed in the informed consent form (ICF) and in this protocol.

### Patient Exclusion Criteria

Participants are excluded from the study if any of the following criteria applies:

1. Have received prior treatment with chemotherapy (except for neoadjuvant/adjuvant chemotherapy), fulvestrant, any investigational-ER-directed therapy (including SERDs and non-SERDs), any PI3K-, mTOR-, or AKT-inhibitor

2. Are currently receiving an investigational drug in a clinical trial or participating in any other type of medical research judged not to be scientifically or medically compatible with this study

3. Have inflammatory breast cancer

4. Patients with known pathogenic germline mutations who are appropriate for treatment with a PARP inhibitor, in regions where these therapies are approved and available, are not eligible for this study.

5. Have visceral crisis, lymphangitic spread within the lung, or any evidence of leptomeningeal disease. Visceral crisis is not the mere presence of visceral metastases but implies severe organ dysfunction as assessed by symptoms and signs, laboratory studies, and rapid progression of the disease

6. Have symptomatic or untreated brain metastasis. Patients with treated brain metastases are eligible for this study if they completed prior therapy (including radiation and/or surgery) ≥28 days prior to first dose of study treatment and are not receiving corticosteroids and/or anticonvulsants for at least 14 days prior to first dose of study treatment, and their disease is asymptomatic and radiographically stable for at least 28 days prior to consent by repeat imaging (repeat imaging should be performed during study screening)

7. Have had major surgery within 14 days prior to randomization

8. Have had wide-field radiotherapy ≤4 weeks (defined as involving ≥25% of the bone marrow), or limited field radiation for palliation ≤1 week prior to randomization. Patients must also have recovered to grade 1 or better from related side effects of such therapy (with the exception of alopecia)

9. Have a serious cardiac condition, such as

a. congestive heart failure

b. New York Heart Association Class III/IV heart disease

c. unstable angina pectoris

d. myocardial infarction within the last 3 months

e. valvulopathy that is severe or moderate, or deemed clinically significant

f. arrhythmias that are symptomatic or require treatment (not including patients with rate-controlled atrial fibrillation)

g. cerebrovascular accident (stroke) within the last 3 months

h. a mean QT interval corrected for heart rate of ≥470 msec on screening ECG, as calculated using the Fridericia's formula at several consecutive days of assessment

i. baseline bradycardia with resting heart rate <60 beats per minute

10. Have serious preexisting medical conditions that, in the judgment of the investigator, would preclude participation in this study

11. Have a history of any other cancer (except nonmelanoma skin cancer or carcinoma in-situ of the cervix), unless in complete remission with no therapy for a minimum of 3 years

12. Have received an autologous or allogeneic stem cell transplant

13. Have active bacterial or fungal infection, or detectable viral infection (for example, human immunodeficiency virus [HIV] or viral hepatitis). Screening is not required for enrollment

14. Pregnant, breastfeeding, or expecting to conceive or father children within the projected duration of the trial, starting with the screening visit through 180 days after the last dose of study intervention

15. Have initiated bisphosphonates or approved RANK ligand (RANK-L) targeted agents (for example, denosumab) <7 days prior to randomization

16. Known allergic reaction against any of the components of the study treatment. Claims

1. A method of treating a cancer comprising:
administering a dose between about 200 mg and about 400 mg of a compound of Formula I: 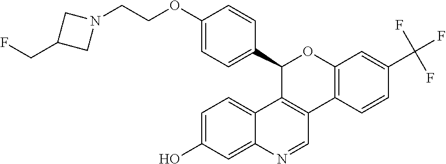 [SMILES: Oc1ccc2c3c(cnc2c1)-c1ccc(C(F)(F)F)cc1O[C@@H]3c1ccc(OCCN2CC(CF)C2)cc1]
or pharmaceutically acceptable salt thereof, to a patient in need of such treatment, at least once a day for at least one week.

2. The method of claim 1, wherein the pharmaceutically acceptable salt is the tosylate salt.

3. The method of claim 1, wherein the dose is about 200 mg, about 300 mg, or about 400 mg.

4. The method of claim 1, wherein the dose is about 400 mg.

5. The method of claim 1, wherein the patient has been identified or diagnosed as having a cancer selected from the group consisting of: breast cancer, ovarian cancer, endometrial cancer, prostate cancer, uterine cancer, gastric cancer, and lung cancer.

6. The method of claim 5, wherein the cancer is HR-positive.

7. The method of claim 6, wherein the HR-positive cancer is ER-positive and HER2-negative.

8. The method of claim 6, wherein the HR-positive cancer is ER-positive and HER2-positive.

9. The method of claim 6, wherein the cancer is metastatic breast cancer (mBC).

10. The method of claim 6, wherein the cancer is advanced breast cancer.

11. The method of claim 6, wherein the cancer is endometrioid endometrial cancer (EEC).

12. A method of treating a cancer according to claim 4, the method further comprising: administering a second therapeutic agent.

13. The method of claim 12, wherein the second therapeutic agent is selected from the group consisting of: abemaciclib, an aromatase inhibitor, everolimus, alpelisib, trastuzumab, and pertuzumab.

14. The method of claim 13 wherein the aromatase inhibitor is selected from the group consisting of: anastrozole, exemestane, and letrozole.

15. The method of claims 12, wherein the second therapeutic agent is abemaciclib.

16. The method of claim 12, wherein the second therapeutic agent is trastuzumab.

17. The method according of claim 12, wherein the compound of Formula I or pharmaceutically acceptable salt thereof and second therapeutic agent are administered with a third therapeutic agent.

18. The method according to claim 17, wherein third therapeutic agent is selected from the group consisting of: an aromatase inhibitor, everolimus, alpelisib, trastuzumab, and pertuzumab and wherein the third therapeutic agent differs from the second therapeutic agent.

19. The method of claim 18, wherein the third therapeutic agent is selected from the group consisting of an aromatase inhibitor and trastuzumab.

20. The method of claim 18, wherein the aromatase inhibitor is selected from the group consisting of anastrozole, exemestane, and letrozole.

21. The method of claim 17, wherein the third therapeutic agent is an antidiarrheal agent.

22. The method of claim 17, wherein the third therapeutic agent is pertuzumab.

23. A method of treating ER+, HER2− breast cancer, the method comprising administering (5R)-)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1), pertuzumab and trastuzumab.

24. A method of treating HR+ and ER+ cancer, the method comprising administering about 400 mg of (5R)-5-[4-[2-[3-(fluoromethyl)azetidin-1-yl]ethoxy]phenyl]-8-(trifluoromethyl)-5H-chromeno[4,3-c]quinolin-2-ol, 4-methylbenzenesulfonic acid (1/1) and abemaciclib, wherein the cancer is selected from the group consisting of HER2-positive breast cancer, HER2-negative breast cancer and endometrioid endometrial cancer (EEC).
